(19) United States
(12) Patent Application Publication
(10) Pub. No.: US 2010/0227412 A1
(43) Pub. Date: Sep. 9, 2010

(54) Simultaneous Detection of Estrogen and Non Estrogen Steroids

(75) Inventor: Blas Cerda, Milford, MA (US)

Correspondence Address: FISH & RICHARDSON PC, P.O. BOX 1022, MINNEAPOLIS, MN, 55440-1022, US

(73) Assignee: PERKINELMER HEALTH SCIENCES, INC., Waltham, MA (US)

(21) Appl. No.: 12/701,161

(22) Filed: Feb. 5, 2010

Related U.S. Application Data
(60) Provisional application No. 61/150,639, filed on Feb. 6, 2009.

(51) Int. Cl. G01N 33/50

(52) U.S. Cl. 436/131

(57) ABSTRACT

Methods for determining the amounts of estrogen and non-estrogen steroids in a sample are provided. The methods employ the selective derivatization of estrogen steroids present in a sample and detection of the molecular ions and fragments of the derivatized estrogens and non-estrogen steroids in the sample. The methods provided herein enable the simultaneous quantification of estrogen and non-estrogen steroids in a sample.

This application claims the benefit of priority of U.S. Provisional Appl. No. 61/150,639, Feb. 6, 2009, which is incorporated by reference in its entirety.

### TECHNICAL FIELD

This disclosure relates to mass spectrometry techniques useful in the determination of the presence and amount of steroids in biological tissues and fluids, e.g., the simultaneous determination of estrogen and non estrogen steroids. The methods described herein can be employed in, e.g., monitoring hormone replacement therapy or the diagnosis of certain disease states or conditions.

### BACKGROUND

Quantification of steroids involved in biological processes is routinely performed for diagnosis of certain disease states and for monitoring hormone replacement therapies. Examples of such steroids include corticosteroids, anabolic steroids, and hormone steroids, such as estrogens, progesterone and testosterone.

Various methods can be employed for the detection of steroid levels in a sample, including immunoassays, high performance liquid chromatography (HPLC) with ultra-violet (UV) fluorescent detection, and liquid chromatography in conjunction with mass spectrometry (LC/MS) and/or tandem mass spectrometry (MS/MS). Techniques using mass spectroscopy for the analysis of multiple steroids in a sample offer many benefits, but can require time consuming pre-analysis purification. In addition, standard protocols can call for first separating estrogen steroids and non-estrogen steroids in to two samples and then analyzing each sample separately for the accurate determination of each type of analyte. This is primarily due to the lower concentration of estrogen steroids in biological samples and difficulties in detecting them, e.g., due to the low sensitivity fluorometric and mass spectrometry detection methods.

Sensitive methods for the simultaneous determination of estrogen and non-estrogen steroids would provide a less expensive and more efficient means for the analysis of steroids.

### SUMMARY

Described herein are materials and methods useful in the detection of estrogen and non-estrogen steroids, e.g., for the simultaneous or sequential determination of estrogen and non-estrogen steroids in a biological sample. In certain instances, 13 or more steroids can be detected simultaneously. Such simultaneous detection offers a cost effective, simple, and efficient means for rapid determination of multiple analytes useful in, e.g., the diagnosis of disease states or conditions and hormone therapy monitoring. The methods described herein can be used with as little as 200 μL of sample.

Provided herein are methods for detecting at least one of each of an estrogen and a non-estrogen steroid in a sample. In certain instances, the method comprises the steps of contacting a sample, with a derivatization agent under conditions suitable to selectively derivative the estrogen steroids present in the sample and using a mass spectrometry technique to detect at least one derivatized estrogen and at least one underivatized non-estrogen steroid in the sample. Such selective derivatization of the estrogen steroids present in a sample enhances detection sensitivity for the resulting derivatized estrogen while minimizing interference from derivatized and underivatized non-estrogen steroids.

Also provided, are methods for enhancing the detection sensitivity for estrogen steroids in a sample containing one or more non-estrogen steroids. In certain instances, the method includes the steps of contacting the sample with a derivatizing agent to give a sample with at least one derivatized estrogen steroid and less than about 10% derivatized non-estrogen steroids and using a mass spectrometry technique to detect the at least one derivatized steroid.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is an illustrative flow chart of one embodiment of the steroid derivatization assay described herein.

FIG. 2a is a chromatogram of a sample containing aldosterone, cortisol, DEAS, 11-deoxycortisol, corticosterone, androstenedione, testosterone, 17-OH progesterone, DHEA, progesterone, estriol, estrone, and estradiol that was not subjected to the selective derivatization methods described herein.

FIG. 2b is a chromatogram of a sample containing aldosterone, cortisol, DEAS, 11-deoxycortisol, corticosterone, androstenedione, testosterone, 17-OH progesterone, DHEA, progesterone, estriol (dansylated), estrone (dansylated), and estradiol (dansylated) that was subjected to the selective derivatization methods described herein.

FIG. 3 is a comparison of the peak area of derivatized and underivatized estrogens using the derivatization conditions and mass spectrometry conditions described herein.

FIG. 4 is a chromatogram of non-estrogen steroids present in a sample subjected to the selective derivatization methods described herein. As can be seen from the chromatogram, no dansylated non-estrogen steroids are detected using the selective derivatization methods described herein.

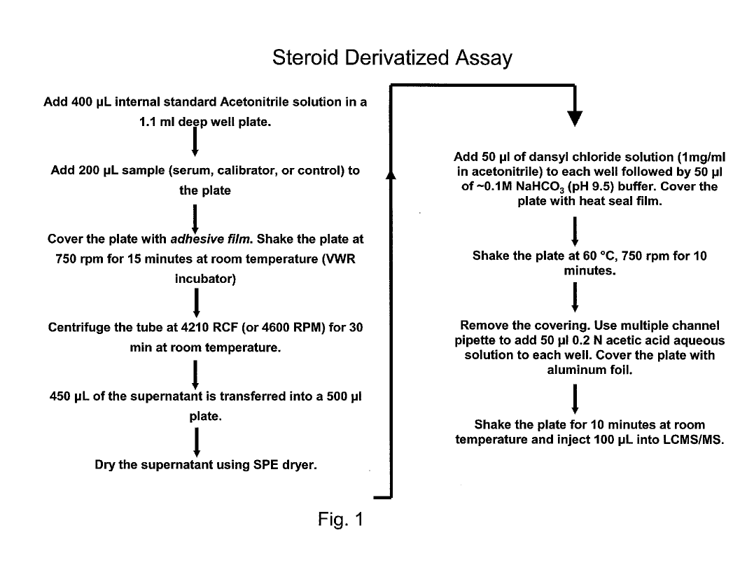

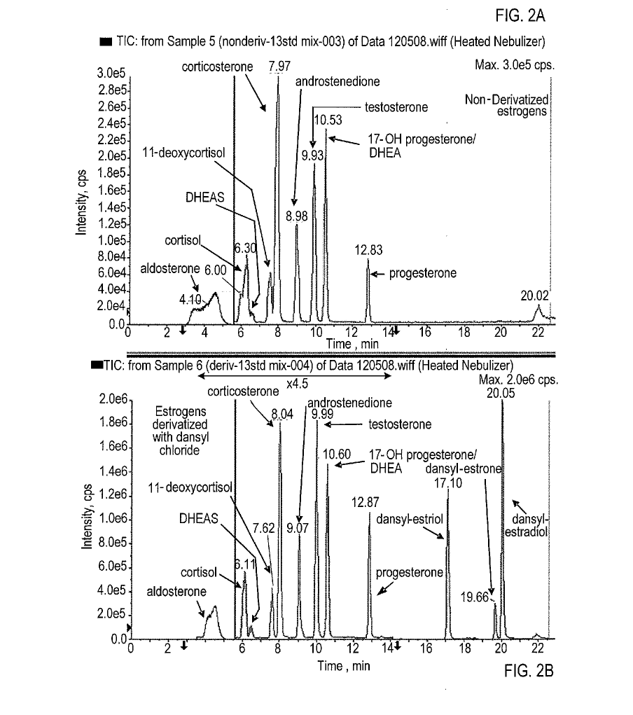

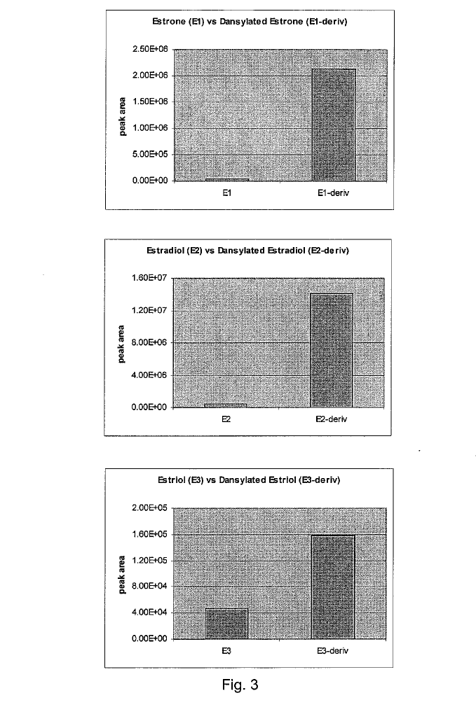

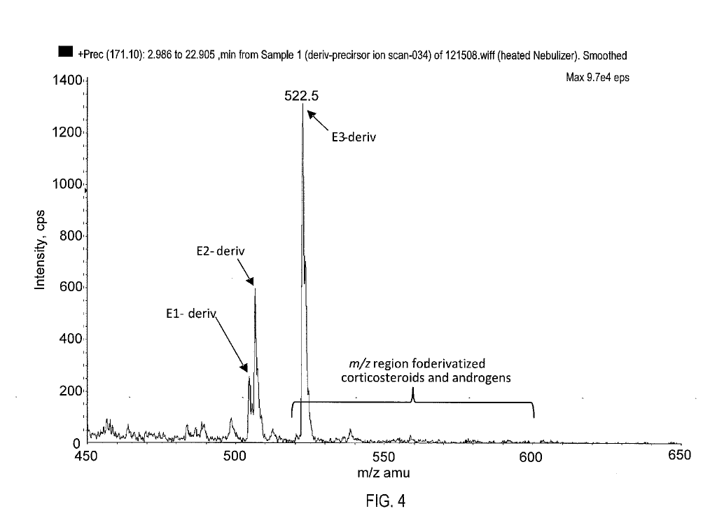

### DETAILED DESCRIPTION

Materials and methods are provided for detecting at least one of each of an estrogen and a non-estrogen steroid in a sample, e.g., a biological sample taken from a patient in a clinical setting. The method provides an efficient and cost effective means for simultaneously determining the levels of multiple steroids in samples as small as 200 μL. The methods provided herein are capable of simultaneously detecting at least one of each of an estrogen and a non-estrogen steroid in a sample. Such non-estrogen steroids include, e.g., mineralocorticoid steroids, glucocorticoid steroids, non-estrogen androgen steroids, progestagen steroids, and synthetic steroids.

In certain instances, the methods described herein are capable of detecting, e.g., simultaneously or sequentially, one or more estrogen steroids (e.g., 16-OH estrone, 2-OH estrone, estrone, equilenin, α-ethinyl-estradiol, 17 β-estradiol, and estriol) and one or more non-estrogen steroids selected from, e.g., dehydroepiandrosterone, dehydroepiandrosterone sulphate, aldosterone, cortisol, corticosterone, 11-deoxycortisol, androstenedione, testosterone, androsterone, isoandrosterone, etiocholanolone, methyl testosterone, estradiol, 17-OH progesterone, progesterone, pregnenolone and allopregnanolone.

A sample to be analyzed can be any sample, including biological and non-biological samples. A sample can be a sample taken from food (e.g., meat or diary). A sample can be a hormone therapy supplement. A sample can be a biological sample such as tissue or fluid (e.g., blood, serum, plasma, urine, or saliva). The biological sample can be from a mammal, such as a human, dog, cat, primate, rodent, pig, sheep, cow, or horse.

Generally, the amount of sample required to practice the methods described herein will vary depending on the nature of the sample, e.g., biological or non-biological, and the analytes of interest. The methods employed herein require as little as about 200 μL of a liquid sample, e.g., blood, urine, or saliva, to be collected. In certain instances, the sample collected is about 200 μL, about 300 μL, about 400 μL, about 500 μL, about 600 μL, about 700 μL, about 800 μL, about 900 μL, or about 1,000 μL.

Solid sample size can very from about 1 mg to about 500 g, about 1 mg to about 400 mg, about 1 mg to about 300 mg, about 1 mg to about 200 mg, about 10 mg to about 100 mg, or about 25 mg to about 75 mg.

A sample can optionally be partially purified prior to analysis by removing some or all interfering and/or extraneous components from the sample. A number of techniques known to those of ordinary skill in the art can be employed depending on the nature of the sample. For example, solid samples (e.g., tissues, tablets, and dried blood spots) can be ground and extracted to free analytes from other solid materials. In such cases, a sample can be extracted (e.g., solid-liquid extractions), centrifuged, filtered, and/or subjected to chromatographic techniques to remove other components. In certain instances, the sample can be purified by adding one or more reagents known to precipitate and/or bind to extraneous and/or interfering components from the sample. For example, conventional reagents, such as acetonitrile, alcohols, KOH, and NaOH, can be used to precipitate serum proteins from serum; after addition of the reagent the serum sample can optionally be centrifuged and the supernatant collected.

In certain instances, an internal standard can be added to the sample prior to or during the sample preparation or purification. Internal standards can be used to monitor estrogen derivatization, sample extraction, and/or sample purification efficiency. For example, derivatization of an analyte, e.g., an estrogen, may, in certain instances, not proceed to completion. The addition of an internal standard to monitor loss of analyte during sample preparation and/or derivatization can be helpful in correcting for the loss of analyte during sample preparation. Analytes can be lost, e.g., during sample purification, incomplete estrogen derivatization, and non-estrogen steroid analyte derivatization. The extent of estrogen derivatization can be determined by comparison of portion of known amount of one or more estrogen internal standards added to the sample that has been derivatized with the portion that has not been derivatized. Likewise, loss of analyte during sample preparation and/or derivatization of non-estrogen steroids can be determined by comparing the amount of the internal standard used for the analyte of interest that is detected after sample preparation and detection. An internal standard can be added to a sample and allowed to equilibrate for a period of time, e.g., 5, 10 15, 20, 25, 30, 60, 120 or more minutes. The equilibration temperature can be from about 10° C. to about 45° C., or any value in between (e.g., 15° C., 25° C., 30° C., 35° C., 37° C., or 44° C.).

An internal standard can be any compound that would be expected to behave under the sample preparation, derivatization, and/or analysis conditions in a manner similar to that of one of more of the analytes of interest. In certain instances, the internal standard can be a close structural analog of the analyte of interest. In certain instances, an isotopically labeled analog of an analyte of interest can be used as an internal standard. Isotopically labeled internal standards can contain one or more isotopes selected from ²H and ¹³C. In certain instances, an isotopic standard can be used for one or more of the analytes of interest. Examples of isotopically labeled standards useful in the procedures described here are illustrated in Table 1 below.

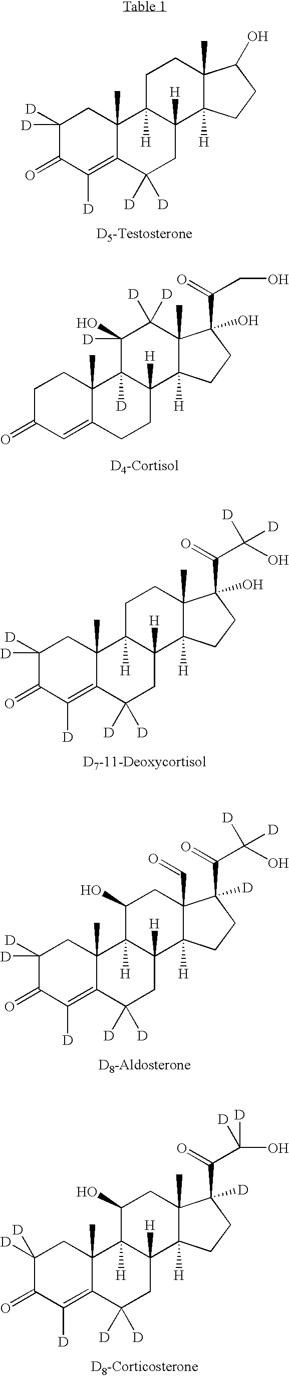

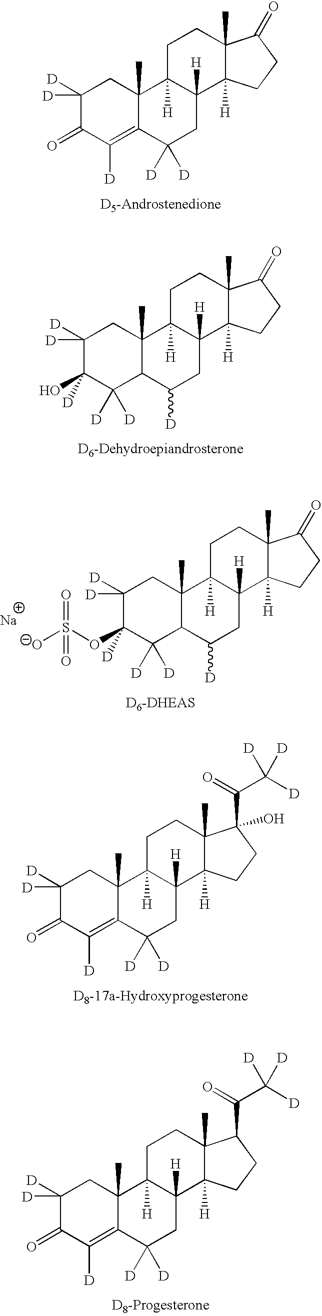

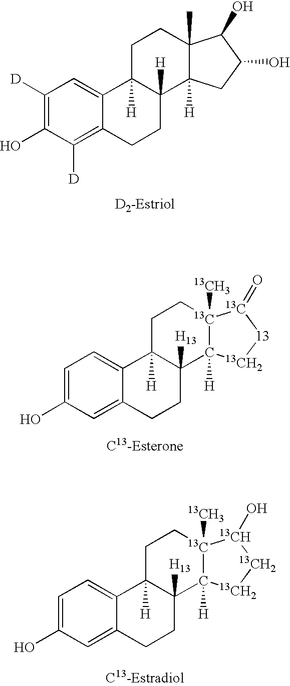

The sample can then be subjected to the derivatization conditions. The derivatization agent can be any agent that selectively reacts with estrogen compounds in the presence of non-estrogen steroids under the derivatization conditions employed. In certain instances, less than about 20%, less than about 15%, less than about 10%, less than about 7%, less than about 5%, less than about 3%, less than about 1%, or substantially none of the non-estrogen steroids present in the sample are derivatized using the estrogen steroid derivatization conditions employed.

The derivatization reagent can be any reagent that that reacts selectively with estrogen steroids in the presence of non-estrogen steroids. In certain instances, the derivatization agent is any reagent that can selectively react with the aromatic ring, e.g., a derivatization reagent that can undergo an electrophilic aromatic substitution reaction, present in an estrogen steroid. In certain instances, the derivatization reagent is any reagent that can selectively react with a phenolic moiety or its salt that may be present in estrogen steroids.

The derivatization agent can be a sulfonyl chloride, an alkyl halide (e.g., benzyl bromide), an isocyanate, or a thioisocyanate. The derivatizing agent can optionally include a basic or acidic moiety, such as an amine or carboxylic acid. In certain instances, the derivatizing agent is 5-(dimethylamino)naphthalene-1-sulfonyl chloride, pyridine-3-sulfonyl chloride, 1,2-dimethylimidazole sulfonyl chloride, naphthalene-1-sulfonyl chloride, dabsyl chloride, or 4-(1H-pyrazol-1-yl)benzenesulfonyl chloride.

A base can be used in the derivatization reaction. The base can be an organic or inorganic reagent. In certain instances, the base has a pK_{b} suitable for selectively deprotonating the phenolic moiety of an estrogen steroid in the presence of non-estrogen steroidal aliphatic alcohols. In certain instances, the pK_{b} of the base employed in the derivatization step is about 8, 6, 5, 4, 3, 2, 1, or 0 or less. In certain instances, the base employed is an alkali or alkaline earth carbonate or bicarbonate, such as Na₂CO₃, K₂CO₃, Li₂CO₃, CaCO₃, MgCO₃, NaHCO₃, KHCO₃, and LiHCO₃. In the examples below NaHCO₃ is used as the base.

In certain instances, acetonitrile is used as the solvent for the derivatization reaction. In general, any solvent can be used that at least some or all of the components of the sample are at least partially soluble in.

The derivatization step can optionally be conducted in a pH buffered solution. The selection of the pH buffering agent is well within knowledge of a person of ordinary skill in the art. The pH of the buffered solution can be between about 8 and about 14, about 8 and about 12, about 8 and about 10, or about 8.5 to about 9.5. In certain instances, the solvent is buffered at a pH of about 9 or about 9.5.

The sample can be reacted with the derivatizing agent until at least a portion or substantially all of the estrogen steroids present in the sample are derivatized. For example, at least 50%, at least 60%, at least 70%, at least 80%, at least 85%, at least 90%, at least 95%, or at least 98% of the estrogen analyte(s) of interest can be derivatized.

The time that the derivatization reagent and the sample remain in contact can be affected by a number of parameters, including the nature of the derivatization reagent employed, the reaction solvent used, and the reaction temperature. The selection of the proper derivatization reagent, reaction time, and reaction solvent is well within the skill of a person of ordinary skill in the art.

In certain instances, the derivatizing reagent and the sample are allowed to react for less than about 60, about 45, about 30, about 25, about 20, about 15, less than about 10, or less than about 5 minutes. The reaction temperature can be about 80° C., 70° C., 60° C., 50° C., 40° C., 30° C., 20° C. or about 10° C. In the examples described below, the sample is allowed to react with the derivatization reagent in buffered solution of acetonitrile for about 3-5 minutes at temperatures between about 45° C. to about 60° C.

After the sample has been subjected to the derivatization reaction, the components of the sample can be separated using liquid chromatography. In certain instances, reverse phase column chromatography, such as using a non-polar stationary phase, e.g., a C-18 column, is used to separate and elute the components of the sample. Extraneous components, such as unreacted materials from the derivatization step or side products can be removed at this step to, e.g., improve analysis efficiency and analysis run time. Analytes that elute from the analytical chromatography column can then be measured by mass spectrometry techniques, such as tandem mass spectrometry.

The analytes can then be introduced into a mass spectrometer. Optionally, the step of separating the analytes of the sample can be combined with the introduction of the analytes into the mass spectrometer by using an LC-MS or LC-MS/MS machine.

The analytes are then subjected to ionization. Various ionization techniques can be used. For example, photoionization, electrospray ionization (ESI), atmospheric pressure chemical ionization (APCI), and electron capture ionization may be used. In the examples below APCI is used to ionize the sample.

Ionization may be performed by utilizing the mass spectrometer in the negative or the positive mode. Factors such as a particular analyte's tendency to give rise to a particular ion form, as is known to those skilled in the art, may make either the negative mode or the positive mode better suited. In the examples below testosterone, DHEA, 17-hydroxyprogesterone, corticosterone, progesterone, 11-deoxycortisol, androstenedione, DHEAS, cortisol, estrone (dansylated), estradiol (dansylated), and estriol (dansylated) are ionized in positive ion mode and aldosterone is ionized in negative ion mode.

MS analysis can be conducted with a single mass analyzer (MS) or a “tandem in space” analyzer, such as a quadropole tandem mass spectrometer (MS/MS). Parent-daughter ion transition monitoring (PDITM) can be used to detect ions generated by ionization and further fragmentation. PDITM includes measurement using mass spectrometry whereby the transmitted mass-to-charge (m/z) range of a first mass separator is selected to transmit a molecular ion (the parent ion or precursor ion) to an ion fragmentor (e.g. a collision cell, photodissociation region, etc.) to produce fragment ions (daughter ions) and the transmitted m/z range of a second mass separator is selected to transmit one or more daughter ions to a detector which measures the daughter ion signal. The combination of parent ion and daughter ion masses monitored can be referred to as the “parent-daughter ion transition” monitored.

In certain instances, PDITM is accomplished by multiple reaction monitoring (MRM). In various embodiments of MRM, the monitoring of a given parent-daughter ion transition comprises using the first mass separator (e.g., a first quadrupole set to detect a parent ion m/z of interest) to transmit the parent ion of interest and using the second mass separator (e.g., a second quadrupole set to detect a daughter ion m/z of interest) to transmit one or more daughter ions of interest. In various embodiments, a PDITM can be performed by using the first mass separator (e.g., a quadrupole set to detect an ion m/z of interest) to transmit parent ions and scanning the second mass separator over a m/z range including the m/z value of the one or more daughter ions of interest.

Parent ions and/or daughter ions corresponding to the derivatized estrogen and non-derivatized non-estrogen steroid analytes can be selected and monitored. Measurement of the intensity of each of the analyte peaks, relative to the corresponding internal standard and/or calibration curves for known concentrations of an analyte of interest can be used to determine the amounts of each analyte in the sample.

A mass spectrometer can be tuned to monitor any known ion and/or precursor ion/product ion transition. Table 2 illustrates certain parent daughter transitions (Transition 1 and Transition 2 below) that can be monitored to detect the corresponding analyte in the sample. One or both transitions can be monitored to detect the corresponding steroid.

TABLE 2

| Steroid (Ionization Mode)* | MW | Transition 1 | Transition 2 |
|---|---|---|---|
| testosterone (+) | 288.2 | 289/109 | 289/97 |
| DHEA (+) | 288.2 | 271/213 | 253/197 |
| 17-hydroxyprogesterone (+) | 330.2 | 331/109 | 331/97 |
| corticosterone (+) | 346.2 | 347/121 | 347/97 |
| progesterone (+) | 314.2 | 315/109 | 315/97 |
| 11-deoxycortisol (+) | 346.2 | 347/109 | 347/97 |
| androstenedione (+) | 286.2 | 287/97 | 287/109 |
| DHEAS (+) | 426.2 | 271/213 | 253/197 |
| cortisol (+) | 362.2 | 363/121 | 363/97 |
| aldosterone (−) | 360.2 | 359/189 | 359/331 |
| estrone (E1) dansylated (+) | 503.3 | 504/171 | 504/203 |
| estradiol (E2) dansylated (+) | 505.3 | 506/171 | 506/203 |
| estriol (E3) dansylated (+) | 521.3 | 522/171 | 522/203 |

Steroid analytes labeled “+” can be detected in positive-ion mode and “−” can be detected in negative-ion mode.

Using the methods described herein, multiple steroids can be measured simultaneously. For example, one or more estrogen steroids and at least 1, 2, 3, 4, 5, 6, 7, 8, 9, 10, 11, 12, 13, 14, 15, or 16 non-estrogen steroids can be detected and/or measured simultaneously.

In certain instances, the methods described herein can be used to measure one or more estrogen steroids and one, two, three, or four androgens. In certain instances, the methods described herein can be used to measure one or more estrogen steroids and one, two, three, or four glucocorticoid steroids. In certain instances, the methods described herein can be used to measure one or more estrogen steroids and one, two, three, or four mineral corticoid steroids.

In certain instances, the methods described herein can be used to measure one or more estrogen steroids, one or more androgen steroids, and one or more glucocorticoid steroids. In certain instances, the methods described herein can be used to measure one or more estrogen steroids, one or more androgen steroids, and one or more mineral corticoid steroids. In certain instances, the methods described herein can be used to measure one or more estrogen steroids, one or more mineral corticoid steroids, and one or more glucocorticoid steroids.

In certain instances, the methods described herein can be used to measure one or more estrogen steroids, one or more mineral corticoid steroids, one or more androgen steroids, and one or more glucocorticoid steroids.

### EXAMPLES

The following examples serve to illustrate the methods described herein without limiting the scope thereof.

### Example 1

### Derivatization of Estrogen Steroids with Dansyl Chloride

### Materials

An optimized LC MS/MS method using an API5000 mass spectrometer and Shimadzu liquid chromatography system was used for all experiments. Samples used are endogenous off-the-clot pooled human serum samples spiked with known amounts of steroids. All peak areas reported are an average of three injections with corresponding % CV values.

### Derivatization Procedure

Before derivatization, the steroids are extracted from serum and dried on a 500 μL polypropylene plate. An exemplary derivatization procedure is as follows:
1. A 60 μL aliquot of a 1 mg/mL dansyl chloride solution in acetonitrile is added to each sample.
2. A 60 μL aliquot of buffer (NaHCO₃ in water) is added to each sample.
3. The plate is heat-sealed and shaken at 750 rpm.
4. The seal is removed and 52 μL of 0.2 M aqueous acetic acid is added to each sample.
5. The plate is covered with foil, shaken at 500 rpm for 10 minutes at room temperature
6. 130 μL of each sample is injected into the LCMS/MS system.

Data collected from the analysis of dansylated estrogen derivatives at different pH levels is shown in Tables 3a-c below (heat-sealed and shaken at 750 rpm for 3 minutes at 60 C). The pH ranges examined 8, 9, 9.5, 10, and 10.5. According to Table 3a-3c, yields of the dansylated estrogens increased with pH. However, detection sensitivity of the resulting dansylated samples decreased at higher pH possibly due to interference from non-estrogen steroids.

TABLE 3a
Estrone-E1

| | |
|---|---|
| 20 pg/mL estrone | 300 pg/mL estrone |

| pH | average area | % CV | average area | % CV |
|---|---|---|---|---|
| pH 8.5 | 3.42E+03 | 11% | 2.81E+04 | 33% |
| pH 9 | 3.67E+03 | 3% | 2.57E+04 | 32% |
| pH 9.5 | 4.13E+04 | 6% | 2.93E+05 | 16% |
| pH 10 | 4.28E+04 | 4% | 3.77E+05 | 11% |
| pH 10.5 | 9.37E+04 | 7% | 5.87E+05 | 13% |

TABLE 3b
Estradiol-E2

| | |
|---|---|
| 20 pg/mL estradiol | 300 pg/mL estradiol |

| pH | average area | % CV | average area | % CV |
|---|---|---|---|---|
| pH 8.5 | 1.61E+04 | 29% | 3.19E+04 | 26% |
| pH 9 | 1.23E+04 | 8% | 3.43E+04 | 41% |
| pH 9.5 | 1.09E+05 | 15% | 2.46E+05 | 17% |
| pH 10 | 1.09E+05 | 10% | 4.20E+05 | 7% |
| pH 10.5 | 1.64E+05 | 1% | 5.59E+05 | 12% |

TABLE 3c
Estriol-E3

| | |
|---|---|
| 20 pg/mL estriol | 300 pg/mL estriol |

| pH | average area | % CV | average area | % CV |
|---|---|---|---|---|
| pH 8.5 | 5.69E+03 | 26% | 3.60E+04 | 15% |
| pH 9 | 6.94E+03 | 11% | 2.97E+04 | 26% |
| pH 9.5 | 3.74E+04 | 6% | 2.86E+05 | 10% |
| pH 10 | 4.04E+04 | 9% | 3.34E+05 | 11% |
| pH 10.5 | 7.85E+04 | 5% | 5.91E+05 | 8% |

Data collected from the analysis of dansylated estrogen derivatives using different concentrations of dansyl chloride is shown in Tables 4a-c below. Three concentrations of the dansyl chloride solution were tried: 0.5, 1 and 2 mg/mL. Yields of the dansylated estrogens increased with concentration. However, detection sensitivity of the resulting dansylated samples decreased at higher concentrations possibly due to interference from non-estrogen steroids.

TABLE 4a
Estrone-E1

| Dansyl Chloride | 20 pg/mL estrone | | 300 pg/mL estrone | |
|---|---|---|---|---|
| Concentration | average area | % CV | average area | % CV |
| 0.5 mg/mL | 3.90E+04 | 18% | 4.19E+05 | 13% |
| 1.0 mg/mL | 9.37E+04 | 7% | 5.87E+05 | 13% |
| 2.0 mg/mL | 9.40E+04 | 10% | 7.06E+05 | 20% |

TABLE 4b
Estradiol-E2

| Dansyl Chloride | 20 pg/mL estradiol | | 300 pg/mL estradiol | |
|---|---|---|---|---|
| Concentration | average area | % CV | average area | % CV |
| 0.5 mg/mL | 9.11E+04 | 24% | 4.42E+05 | 9% |
| 1.0 mg/mL | 1.64E+05 | 1% | 5.59E+05 | 12% |
| 2.0 mg/mL | 3.21E+05 | 17% | 9.77E+05 | 5% |

TABLE 4c
Estriol-E3

| Dansyl Chloride | 20 pg/mL estriol | | 300 pg/mL estriol | |
|---|---|---|---|---|
| Concentration | average area | % CV | average area | % CV |
| 0.5 mg/mL | 3.26E+04 | 8% | 3.09E+05 | 19% |
| 1.0 mg/mL | 7.85E+04 | 5% | 5.91E+05 | 8% |
| 2.0 mg/mL | 7.95E+04 | 6% | 6.80E+05 | 15% |

Data collected from the analysis of dansylated estrogen derivatives using different to reaction times for the dansylation reaction is shown in Tables 5a-c below. Derivatization using dansyl chloride was examined at different times points. Data is shown for reactions run at pH 9.5 for 10 and 20 minutes in Tables 5a-c.

TABLE 5a
Estrone-E1

| | |
|---|---|
| 8 pg/mL estrone | 300 pg/mL estrone |

| Time | average area | % CV | average area | % CV |
|---|---|---|---|---|
| 10 min | 6.14E+04 | 6% | 6.06E+05 | 12% |
| 20 min | 6.96E+04 | 9% | 5.74E+05 | 9% |

TABLE 5b
Estradiol-E2

| | |
|---|---|
| 8 pg/mL estradiol | 300 pg/mL estradiol |

| Time | average area | % CV | average area | % CV |
|---|---|---|---|---|
| 10 min | 1.63E+05 | 6% | 8.21E+05 | 11% |
| 20 min | 1.77E+05 | 10% | 7.52E+05 | 8% |

TABLE 5c
Estriol-E3

| | |
|---|---|
| 8 pg/mL estriol | 300 pg/mL estriol |

| Time | average area | % CV | average area | % CV |
|---|---|---|---|---|
| 10 min | 4.10E+04 | 6% | 6.10E+05 | 7% |
| 20 min | 5.30E+04 | 37% | 5.44E+05 | 7% |

### Example 2

### Derivatization of Estrogen Steroids

1. A 60 μL aliquot of a 1 mg/mL dansyl chloride solution in acetonitrile is added to each sample.
2. A 60 μL aliquot of pH 9.5 buffer (NaHCO₃ in water) is added to each sample.
3. The plate is heat-sealed and shaken at 750 rpm for 10 minutes at 60° C.
4. The seal is removed and 52 μL, of 0.2 M aqueous acetic acid is added to each sample.
5. The plate is covered with foil, shaken at 500 rpm for 10 minutes at room temperature.
6. 130 μL of each sample is injected into the LCMS/MS system.

A number of embodiments of methods for detecting multiple steroids have been described herein. Nevertheless, it will be understood that various modifications may be made without departing from the spirit and scope of this disclosure.

What is claimed is:

1. A method for detecting at least one of each of an estrogen and a non-estrogen steroid in a sample, comprising:
a) contacting said sample with a derivatizing agent to give at least one derivatized estrogen steroid; and
b) using a mass spectrometry technique to detect at least one derivatized estrogen steroid and at least one non-estrogen steroid in said sample.

2. The method of claim 1, wherein said mass spectrometry technique comprises an LC-MS/MS technique.

3. The method of claim 1, further comprising purifying the sample prior to said contacting said sample with a derivatizing agent.

4. The method of claim 3, wherein said sample is a biological sample.

5. The method of claim 4, wherein said biological sample is blood, plasma, serum, urine, or saliva.

6. The method of claim 4, wherein said purifying said biological sample comprises:
a) adding one or more organic solvents;
b) subjecting said sample to centrifugation to give a biological sample supernatant; and
c) collecting said biological sample supernatant.

7. The method of claim 6, wherein said one or more organic solvents is acetonitrile or an alcohol.

8. The method of claim 1, further comprising adding one or more internal standards to said sample prior to the step of contacting said sample with a derivatizing agent.

9. The method of claim 8, wherein said one or more internal standards is one or more isotopically labeled compounds.

10. The method of claim 1, wherein said derivatizing agent is a sulfonyl chloride.

11. The method of claim 8, wherein said sulfonyl chloride is selected from the group consisting of 5-(dimethylamino)naphthalene-1-sulfonyl chloride, pyridine-3-sulfonyl chloride, 1,2-dimethylimidazole sulfonyl chloride, naphthalene-1-sulfonyl chloride, dabsyl chloride, and 4-(1H-pyrazol-1-yl)benzenesulfonyl chloride.

12. The method of claim 1, wherein said contacting occurs in solution at a pH of about 8 to about 10.

13. The method of claim 1, wherein said contacting occurs in a solution at a pH of about pH about 8.5 to about 9.5.

14. The method of claim 12, wherein said solution further comprises a pH buffering agent capable of buffering said solution at a pH of about 8 to about 10.

15. The method of claim 14, wherein said pH buffering agent comprises one more of the species selected from the group consisting of alkali carbonate, alkaline earth carbonate, alkali bicarbonate, and alkaline earth carbonate.

16. The method of claim 12, wherein said derivatizing agent is a sulfonyl chloride.

17. The method of claim 16, wherein said contacting occurs at a temperature of about 25° C. to about 80° C.

18. The method of claim 14, wherein said contacting occurs at a temperature of about 45° C. to about 60° C.

19. The method of claim 16, wherein said biological sample is contacted with said sulfonyl chloride for 10 minutes or less.

20. The method of claim 1, wherein said at least one non-estrogen steroid is selected from the group consisting of: dehydroepiandrosterone, dehydroepiandrosterone sulphate, aldosterone, cortisol, 11-deoxycortisol, androstenedione, testosterone, estradiol, 17-OH progesterone, progesterone, and allopregnanolone.

21. The method of claim 1, wherein said at least one estrogen steroid is selected from the group consisting of 16-OH estrone, 2-OH estrone, estrone, and estriol.

22. The method of claim 1, wherein said sample is 400 μL or less.

23. The method of claim 1, wherein said biological sample is blood, plasma, serum, urine, or saliva.

24. The method of claim 1, wherein said sample is about 200 μL to about 500 μL.

25. The method of claim 1, wherein said sample comprises at least one estrogen steroid and at least two non-estrogen steroids.

26. A method for determining the amount of at least one of each of an estrogen steroid and a non-estrogen steroid in a biological sample, comprising:
a) contacting said biological sample with acetonitrile and one or more isotopically labeled internal standards;
b) subjecting said biological sample to centrifugation to give a biological sample supernatant;
c) contacting said biological sample supernatant with a sulfonly chloride, wherein said contacting occurs in a pH buffered solution at a pH of about 8.5 to about 9.5, to give at least one derivatized estrogen steroid;
d) using an LC-MS/MS technique to detect said at least one derivatized estrogen steroid and at least one non-estrogen steroid in said biological sample.

27. The method of claim 26, wherein said biological sample supernatant is evaporated prior to contacting said biological sample supernatant with said sulfonyl chloride.

28. The method of claim 26, wherein said sulfonyl chloride is 5-(dimethylamino)naphthalene-1-sulfonyl chloride.

29. The method of claim 26, wherein said contacting occurs from about 7 minutes to about 15 minutes.

30. The method of claim 26, wherein said method is used to determine the amount at least one of each of an estrogen steroid and a non-estrogen steroid in a plurality of biological samples.

31. The method of claim 26, wherein said LC-MS/MS technique comprises the use of a triple quadrupole instrument in multiple reaction monitoring ion transitions having m/z values of one or more of 289/109 and 289/97 for testosterone; 271/213 and 253/197 for DHEA; 331/109 and 331/97 for corticosterone; 315/109 and 315/97 progesterone; 347/109 and 347/97 for 11-deoxycortisol; 271/213 and 253/197 for DHEAS; 363/121 and 363/97 for cortisol; 504/171 and 504/203 for estrone; 506/171 and 506/203 for estradiol; 522/171 and 522/203 for estriol; and 359/189 and 359/331 for aldosterone.

32. A method for determining the amount of an estrogen and at least one steroid selected from the group consisting of a mineralocorticoid steroid, a glucocorticoid steroid, an androgen steroid, a progestin steroid, and a synthetic steroid in a sample, comprising:
a) contacting said sample with a derivatizing agent to give at least one derivatized estrogen steroid; and
b) using a mass spectrometry technique to detect said derivatized estrogen steroid and said at least one steroid in said sample.

33. The method of claim 32, wherein said at least one steroid is a mineralocorticoid steroid.

34. The method of claim 32, wherein said at least one steroid is a glucocorticoid steroid.

35. The method of claim 32, wherein said at least one steroid is an androgen steroid.

36. The method of claim 32, wherein said at least one steroid is a progestin steroid.

37. The method of claim 32, wherein said at least one steroid is a synthetic steroid.

38. The method of claim 32, wherein said at least one steroid is 2 or more steroids.

39. A method for enhancing detection sensitivity for at least one or more estrogen steroids in a sample comprising at least one estrogen steroid and at least one non-estrogen steroid selected from the group consisting of dehydroepiandrosterone, dehydroepiandrosterone sulphate, aldosterone, cortisol, 11-deoxycortisol, androstenedione, testosterone, 17-OH progesterone, progesterone, and allopregnanolone, comprising:
c) contacting said sample with a derivatizing agent, to give a sample with at least one derivatized estrogen and less than about 10% derivatized non-estrogen steroids; and
d) using a mass spectrometry technique to detect at least one derivatized estrogen steroid.

40. The method of claim 39, wherein said contacting occurs under conditions wherein less than 5% of said non-estrogen steroids are derivatized.

41. The method of claim 39, wherein said contacting occurs under conditions wherein less than 3% of said non-estrogen steroids are derivatized.

42. The method of claim 39, wherein said derivatization agent is a sulfonyl chloride selected from the group consisting of 5-(dimethylamino)naphthalene-1-sulfonyl chloride, pyridine-3-sulfonyl chloride, 1,2-dimethylimidazole sulfonyl chloride, naphthalene-1-sulfonyl chloride, and 4-(1H-pyrazol-1-yl)benzenesulfonyl chloride.

43. The method of claim 39, wherein said contacting occurs in a solution at a pH of about 9.5.

44. The method of claim 41, wherein said solution further comprises a pH buffering agent capable of buffering said solution at a pH of about 8 to about 10.

45. The method of claim 42, wherein said contacting occurs at a temperature of about 50° C. to about 70° C.
